SEQUENCE LISTING (4 sequences)

SEQ ID NO: 1 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 2 - DNA, 725 bp, Homo sapiens
gtcctgtggacagctcacctagctgcaatggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcct

SEQ ID NO: 3 - protein, 216 aa, Rattus norvegicus
MAADSQTPWLLTFSLLCLLWPQEAGALPAMPLSSLFANAVLRAQHLHQLAADTYKEFERAYIPEGQRYSIQNAQAAFCFSETIPAPTGKEEAQQRTDMELLRFSLLLIQSWLGPVQFLSRIFTNSLMFGTSDRVYEKLKDLEEGIQALMQELEDGSPRIGQILKQTYDKFDANMRSDDALLKNYGLLSCFKKDLHKAETYLRVMKCRRFAESSCAF

SEQ ID NO: 4 - DNA, 6016 bp, adeno-associated virus 2
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaaagctgatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

(19) United States
(12) Patent Application Publication
(10) Pub. No.: US 2009/0105148 A1
(43) Pub. Date: Apr. 23, 2009

(54) COMPOSITIONS AND METHODS FOR TREATING MYOCARDIAL INFARCTION

(75) Inventors: Ryuichi Aikawa, New South Wales (AU); Douglas W. Losordo, Chicago, IL (US)

Correspondence Address: EDWARDS ANGELL PALMER & DODGE LLP, P.O. BOX 55874, BOSTON, MA, 02205, US

(73) Assignee: Caritas St. Elizabeth Medical Center of Boston, Inc., Boston, MA (US)

(21) Appl. No.: 12/294,081

(22) PCT Filed: Mar. 23, 2007
(86) PCT No.: PCT/US07/07247

§ 371 Date: Sep. 23, 2008

Related U.S. Application Data
(60) Provisional application No. 60/785,587, filed on Mar. 23, 2006.

(51) Int. Cl. A61K 48/00; A61P 9/00; A61K 38/18; C12N 5/10

(52) U.S. Cl. 514/12; 514/44; 435/366

(57) ABSTRACT

The invention features compositions and methods that are useful for preventing or treating a cardiac disease or for promoting cardiac health following a myocardial infarction. The invention further features compositions and methods for promoting angiogenesis, cell proliferation, and/or decreasing apoptosis in muscle tissue, such as cardiac tissue. The invention provides for the expression of human growth hormone in cardiac muscle following a myocardial infarction.

### CROSS REFERENCE TO RELATED APPLICATIONS

This application claims the benefit of U.S. Provisional Patent Application Ser. No. 60/785,587 filed on Mar. 23, 2006, which is hereby incorporated by reference in its entirety.

### STATEMENT OF RIGHTS TO INVENTIONS MADE UNDER FEDERALLY SPONSORED RESEARCH

This work was supported by the following grant from the National Institutes of Health, Grant No: HL 53354. The government may have certain rights in the invention.

### BACKGROUND OF THE INVENTION

Over 13 million people worldwide have experienced one or more myocardial infarctions (MI)(acute heart attack), and more than 1.2 million Americans will have a new or recurrent coronary attack this year alone. Moreover, heart disease remains the leading cause of death in the United States. The cardiomyocyte has been considered a terminally differentiated cell with no proliferative capacity; therefore, it has been presumed that a damaged myocardium has no regenerative capacity. For this reason, most experimental and clinical studies for the treatment of cardiomyopathy and heart failure have focused on limiting the infarct size or preserving cardiac function in failing hearts. Improved therapeutic compositions and methods for the treatment of cardiac conditions, such as cardiac myocardial infarction, are urgently required.

### SUMMARY OF THE INVENTION

The present invention features compositions and methods for treating or preventing an ischemic disease, especially an ischemic muscle disease, or a cardiac disease in a tissue of a subject. The invention is based, at least in part, on the observation that transduction of cardiomyocytes with a recombinant adeno-associated viral (rAAV) vector that expresses human growth hormone increases cell proliferation, increases angiogenesis, reduces apoptosis and/or increases function in a cardiac tissue following myocardial infarction.

A feature of the invention includes a method of increasing angiogenesis or cell proliferation in a muscle tissue or a cardiac tissue in a subject in need thereof by administering to the subject an effective amount of a recombinant adeno-associated viral vector expressing growth hormone or a fragment or variant thereof, wherein the administration of the viral vector expressing the growth hormone increases angiogenesis.

A feature of the invention includes a method of decreasing apoptosis in a muscle tissue or a cardiac tissue in a subject in need thereof by administering to the subject an effective amount of a recombinant adeno-associated viral vector expressing growth hormone or a fragment or variant thereof, wherein the administration of the viral vector expressing the growth hormone decreases apoptosis.

A feature of the invention includes a method of increasing muscle function or cardiac function in a subject in need thereof by administering to the subject an effective amount of a recombinant adeno-associated viral vector expressing growth hormone or a fragment or variant thereof, wherein the administration of the viral vector expressing the growth hormone increases cardiac function.

A feature of the invention further includes a method for treating ischemic disease and cardiac disease function in a subject in need thereof by administering to the subject an effective amount of a recombinant adeno-associated viral vector expressing growth hormone or a fragment or variant thereof, wherein the administration of the viral vector expressing the growth hormone ameliorates ischemic or cardiac disease. Cardiac diseases include myocardial infarction, cardiac ischemia, cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure. Ischemic diseases include pathologies related to a chronic and/or acute reduction in the level of oxygen available to a tissue. Ischemic diseases include, but are not limited to, muscle ischemia, critical limb ischemia, myocardial infarction, and stroke.

In a feature of the invention, the growth hormone is matched to the subject in need of therapy. In an embodiment, the subject is a human, and the adeno-associated viral vector expresses human growth hormone. In a feature of the invention, the effect of administration of the viral vector is sustained.

A feature of the invention includes a recombinant muscle cell, such as a cardiac cell, comprising a recombinant adeno-associated viral vector encoding growth hormone or a fragment or variant thereof.

A feature of the invention includes the use of the recombinant adeno-associated viral vector encoding growth hormone or a fragment or variant thereof for use in a medicament for the treatment of cardiac disease or ischemic disease. The invention further includes the viral vectors in kits.

Other features and advantages of the invention will be apparent from the detailed description, and from the claims.

### DEFINITIONS

By “ameliorate” is meant decrease, suppress, attenuate, diminish, arrest, or stabilize the development or progression of a disease.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and even more preferably a 50% or greater change in expression levels.

By “angiogenesis” is meant the formation of neovessels from the endothelium of preexisting vessels.

By “angiogenic factors and mitogens” is meant acidic and basic fibroblast growth factors (aFGF and bFGF), vascular endothelial growth factor (VEGF-1), VEGF165, epidermal growth factor (EGF), transforming growth factor α and β (TGF-α and TFG-β), platelet-derived endothelial growth factor (PD-ECGF), platelet-derived growth factor (PDGF), tumor necrosis factor α (TNF-α), hepatocyte growth factor (HGF), insulin like growth factor-1 (IGF-1), erythropoietin, colony stimulating factor (CSF), macrophage-CSF (M-CSF), granulocyte/macrophage CSF (GM-CSF), angiopoetin-1 (Ang1) and nitric oxidesynthase (NOS); and functional fragments thereof. Muteins or functional fragments of a mitogen may be used as long as they maintain at least a portion of the activity of the corresponding full-length peptide. Angiogenic factors and mitogens can be delivered as peptides or using rAAV vectors for expression.

By “cardiac disease” is meant an event or disorder of the cardiovascular system that affects the heart. Non-limiting examples of cardiovascular conditions affecting the heart include atherosclerosis, primary myocardial infarction, secondary myocardial infarction, angina pectoris (including both stable and unstable angina), congestive heart failure, sudden cardiac death, cerebral infarction, restenosis, syncope, ischemia, reperfusion injury, vascular occlusion, carotid obstructive disease, transient ischemic attack, and the like.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control cells or tissues” is meant cells or tissues not treated with a growth hormone expressing rAAV of the instant invention. Control cells or tissues may be untreated. Alternatively, control cells or tissues may be mock treated with a rAAV vector expressing a gene product (e.g., P-galactosidase) that has no detectable effect of angiogenesis, cell proliferation, apoptosis, or any of the other endpoints claimed herein. Cells or tissues can also be mock treated with buffers and/or inert carriers such as normal saline. Control cells or tissues provide a useful baseline in determining the effect of therapeutic interventions. For example, if an intervention increases an endpoint by 10%, the value of the endpoint is 110% of the control value. Control cells or tissues can be in a separate tissue or animal. Similarly, if an intervention decreases an endpoint by 10%, the value of the endpoint is 90% of the control value. An intervention can increase or decrease an endpoint by about 20%, 30%, 40%, 50%, 60%, 70%, 80%, 90% or 100%. Alternatively, control cells or tissue can be adjacent to treated cells or tissue, but far enough from the treatment site to obtain any benefit from treatment.

By “disease” is meant any condition or disorder that damages or interferes with the normal function of a cell, tissue, or organ.

By “effective amount” is meant an amount sufficient to prevent, treat, or ameliorate a disease or disorder in a subject.

By “fragment” is meant a portion of a polypeptide or nucleic acid molecule having the biological function of the full-length polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “growth hormone” (GH) is meant a polypeptide or fragment thereof having at least 65% amino acid sequence identity to a human growth hormone, where expression of the polypeptide in an ischemic tissue increases angiogenesis, cell proliferation, decreases cell death, or increases organ function. The growth hormone family of proteins includes structurally and functionally related genes and proteins commonly called growth hormones including, but not limited to, the following exemplary polypeptides: human growth hormone (GenBank Accession No. P01241, SEQ ID NO. 1); rat growth hormone (GenBank Accession No. NP_{—}001030020, SEQ ID NO. 3); mole rat growth hormone (GenBank Accession No. CAA06716.1); mouse growth hormone (GenBank Accession No. NP_{—}032143.1); feline growth hormone (GenBank Accession No. NP_{—}001009337.1); canine growth hormone (GenBank Accession No. NP_{—}001003168.1); horse NP_{—}001075417.1); pig growth hormone (GenBank Accession No. AAS89356.1); and rabbit growth hormone (GenBank Accession No. P46407). The species of growth hormone used is preferably selected based on the species to be treated.

By “ischemic disease” is meant a pathology related to a chronic and/or acute reduction in the level of oxygen available to a tissue. Ischemic diseases include, but are not limited to, muscle ischemia, critical limb ischemia, myocardial infarction, and stroke.

By “isolated nucleic acid molecule” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “muscle” or “muscle tissue” is meant skeletal muscle, smooth muscle, and/or cardiac muscle. “Striated muscle” includes cardiac and skeletal muscle.

The term “obtaining” as in “obtaining a rAAV-GH vector” refers to purchasing, synthesizing or otherwise procuring the rAAV vector.

By a “plurality of sites at one time” is meant that at least 2, 3, 4, 5, 6, 7, 8, 9, or 10 injections are made into the muscle tissue as part of a single dose of the rAAV of the invention. The injections can be close together in a single tissue (see, e.g., FIG. 1A). Alternatively, in the case of systemic ischemia with peripheral vascular damage associated with diabetes or other disease, the injections can be made at multiple sites in the subject. The injections are all administered within about an hour, preferably within about 30 minutes, preferably within about 15 minutes.

By “polypeptide” is meant any chain of amino acids, regardless of length or post-translational modification.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “subject” is meant a mammal, including, but not limited to, a human or non-human mammal, such as a bovine, equine, canine, ovine, or feline.

By “sustained” is meant that the effect of the injection of the adeno-associated viral vector can be observed for an extended period after the last administration. For example, sustained can be understood to mean for at least 8, 10, 12, 14, 16, 18, 20, 22, or 24 weeks after the last administration of the viral vector.

By “transgenic” is meant any cell that includes a DNA sequence that is inserted by artifice into a cell and becomes part of the genome of the organism which develops from that cell, or part of a heritable extra chromosomal array. As used herein, transgenic organisms may be either transgenic vertebrates, such as domestic mammals (e.g., sheep, cow, goat, or horse), mice, or rats, transgenic invertebrates, such as insects or nematodes, or transgenic plants.

By “treat” is meant decrease, suppress, attenuate, diminish, arrest, or stabilize the development or progression of a disease.

By “variant” is meant a naturally or non-naturally occurring nucleotide or amino acid sequence that is distinct from the published sequence, such as the sequence in GenBank, in which the variant maintains at least a portion of the desirable properties of the protein or amino acid of the published sequence. Variants may include mutations and/or truncations. Truncations produce fragments that have sequences removed at one or both ends. Variants may differ from the published sequence by about 20%, 15%, 10%, 7%, 5%, 3%, 2%, or 1%.

Terms “a”, “an”, and “the” are understood to be either singular or plural unless otherwise obvious from context.

By “or” is meant to be inclusive unless otherwise obvious from context.

As used herein, ranges are understood to include all values within the range. For example, 1 to 50 is understood to mean 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50. A series of values are understood to represent a range, and thereby all of the values within the range.

### BRIEF DESCRIPTION OF THE DRAWINGS

FIGS. 1A-F demonstrate long-term gene expression from an adenoviral vector in heart. FIG. 1A is a schematic of the rAAV vector injection sites in the myocardium. FIGS. 1B and 1C show tissue sections of heart 4(B) and 22(C) weeks after injection with rAAV-LacZ. FIG. 1D shows a graph of the expression of rAAV-GH in serum measured by ELISA at 0, 4, 10, and 20 W (weeks) after injection. (n=7). FIG. 1E is a schematic of the rAAV vectors used in the methods herein and PCR primers used to amplify the rAAV vector and LacZ sequences. FIG. 1F is an agarose gel showing PCR products amplified from lane 1. PBS-injected heart (control); lane 2. rAAV-LacZ injected heart; or lane 3. rAAV-GH injected heart.

FIGS. 2A to C show a series of graphs demonstrating serial changes in the echocardiographic parameters of A. left ventricular diameter in diastole (LVDd); B. left ventricular diameter in systole (LVDs); and C. fractional shortening (FS) from baseline (day 5 after injection) to 22 weeks after injection. Solid lines indicate rats treated with rAAV-GH vectors and dotted lines indicate rats treated with control lacZ vectors. (n=8). *P<0.01, **P<0.05 vs. control lacZ group.

FIGS. 3A and B are immunohistochemically stained sections of heart injected with (A)rAAV-LacZ or (B)rAAV-GH stained with isolectin to reveal capillaries at 22 weeks. FIG. 3C shows a graph of capillary density in rats receiving rAAV-lacZ or rAAV-GH as determined by immunohistochemical analysis at 22 weeks. (n=8)*p<0.01 vs. control lacZ.

FIGS. 4A to D show a series of graphs of mRNA expression of the angiogenic factors A. eNOS; B. VEGF-A; C. bFGF; and D. Ang-1 in rAAV-infected hearts as determined by quantitative real-time PCR, expressed as normalized ratio to GAPDH (n=6) 4 weeks after injection. *P<0.01 vs. lacZ group.

FIG. 5A is a section from a rAAV-LacZ-infected heart stained with α-actinin, TUNEL stain, and DAPI at 22 weeks. FIG. 5B shows a graph of TUNEL staining per 10⁵ cells 22 weeks after injection of rAAV vectors. (n=8)*P<0.05 vs. control lacZ group.

FIGS. 6A and B are sections from rAAV-LacZ-infected (A) and rAAV-GH-infected (B) hearts stained with α-actinin, Ki-67, and DAPI at 22 weeks after injection. FIG. 6C shows a graph of Ki-67 positive cell staining per 10⁵ cells 22 weeks after injection of rAAV vectors. (n=8)*P<0.05 vs. control.

FIG. 7 shows western blots of total and phosphorylated proteins involved in apoptosis in lysates prepared from hearts injected with rAAV-LacZ or rAAV-GH.

FIGS. 8A and B show graphs of mRNA expression of p53 and p21 assessed by quantitative real-time PCR and expressed as normalized ratio to GAPDH 22 weeks after injection of rAAV vectors. (n=6)*P<0.01 vs. control.

### DETAILED DESCRIPTION OF THE INVENTION

The present invention features compositions and methods for treating or preventing an ischemic disease, especially an ischemic muscle disease, or a cardiac disease in a tissue of a subject. The invention is based, at least in part, on the observation that transduction of cardiomyocytes with a recombinant adeno-associated viral (rAAV) vector that expresses human growth hormone increases cell proliferation, increases angiogenesis, reduces apoptosis and/or increases function in a cardiac tissue following myocardial infarction.

### Growth Hormone

Human growth hormone (hGH, GenBank Accession Nos. amino acid P01241 (SEQ ID NO: 1); nucleotide BC075013 (SEQ ID NO: 2)) is a single chain polypeptide of 191 amino acids that has been characterized as an important regulator of postnatal somatic growth. It has been demonstrated that administration of growth hormone significantly improves the cardiac function of dilated cardiomyopathy and heart failure in clinical and animal studies. Growth hormone expression results in a variety of cell-protective mechanisms, such as its ability to evoke angiogenesis, to enhance the permeability of blood capillaries, and to inhibit apoptosis in post-infarction heart failure. It has been shown that growth hormone binds to its receptor and activates Janus activated kinase (JAK)-2 leading to the activation of phosphoinositide-3-kinase (PI3K)/Akt and STAT3 pathways. Khan et al. (J Gerontol A Biol Sci Med Sci 2001; 56: B364-371) reported that growth hormone administration to aged rats increased coronary artery blood flow and cardiac capillary density in heart.

Growth hormone delivery by direct protein administration requires repeated dosing. This type of administration to the heart is impractical. Gene therapy represents a promising approach for the treatment of many diseases, including inherited heart diseases, cardiomyopathies, and congestive heart failure, and the potential for sustained delivery or a protein therapeutic. See, for example, Nabel E. G (1995) Circulation 91:541-548. Work by Jayasankar et al., (J. Mol. Cell. Cardiol. 36:531-538) shows that recombinant adenoviral (rAd) vectors can efficiently transduce cardiomyocytes in vivo to express ding the potassium channel, sarcoplasmic calcium ATPase-2A, and phospholamban. Long-term expression of genes in cardiomyocytes has not been obtained with adenoviral vectors. To date there have been no reports showing the effect or mechanism of long term growth hormone expression on cardiomyocyte protection in post-myocardial infarction heart failure. Adenoviral vectors are known to induce an immune response upon repeat administration, making the method less useful in the clinic. Designing a delivery system with low cytotoxicity and cardiac-specific gene expression has been a central goal of cardiac gene therapy.

Recombinant adeno-associated virus (rAAV) can be used as a gene transfer vector for heart diseases (Su et al., Proc. Natl. Acad. Sci. USA, 97:13801-13806, 2000; Melo et al., Circulation, 105:602-607, 2002; Hoshijima et al., Nat, Med., 8:864-871, 2002). The small size and physical stability of rAAV make it advantageous for in vivo use, and transgene expression can persist long-term in a wide range of tissues including heart and skeletal muscle (Snyder et al., Hum. Gene Ther., 8:1891-1900, 1997; Fisher et al., Nat. Med., 3:306-312, 1997; Aikawa et al., J. Biol. Chem., 277:18979-18985, 2002).

A preferred viral gene delivery system with low cytotoxicity is provided by vectors derived from a non-pathogenic human parvovirus, i.e., recombinant adeno-associated virus (rAAV). The small size and physical stability of these vectors can be advantageous for in vivo use. Transgene expression from rAAV vectors can persist in a wide range of tissues. Moreover, there is no evidence of cell damage from inflammation after rAAV administration to the liver, skeletal muscle, brain, and heart. Accordingly, rAAV vectors have been recognized as suitable vectors for systemic and local long-term delivery of gene therapy for clinical diseases.

As reported in more detail below, rAAV is capable of transducing cardiac myocytes and the persistent long-term expression of human growth hormone (hGH) by rAAV in the heart has cardioprotective effects following myocardial infarction, demonstrating that the expression of hGH by rAAV may be used for the prevention and/or treatment of cardiac disease. Overexpression of hGH by rAAV was shown to significantly improve cardiac function by promoting angiogenesis and cell proliferation, and protecting cardiomyocytes from apoptosis induced by myocardial infarction. The amino acid sequence of hGH is 64% identical to rat growth hormone (GenBank Accession No. NP_{—}001030020 (SEQ ID NO: 3), demonstrating that variation in the sequence of growth hormone can be tolerated from the native protein of the species to be treated within the scope of the invention. The use of the coding sequence of growth hormone for the species to be treated is preferred. Methods to identify possible variations that are useful in the methods of the invention are within the ability of those skilled in the art.

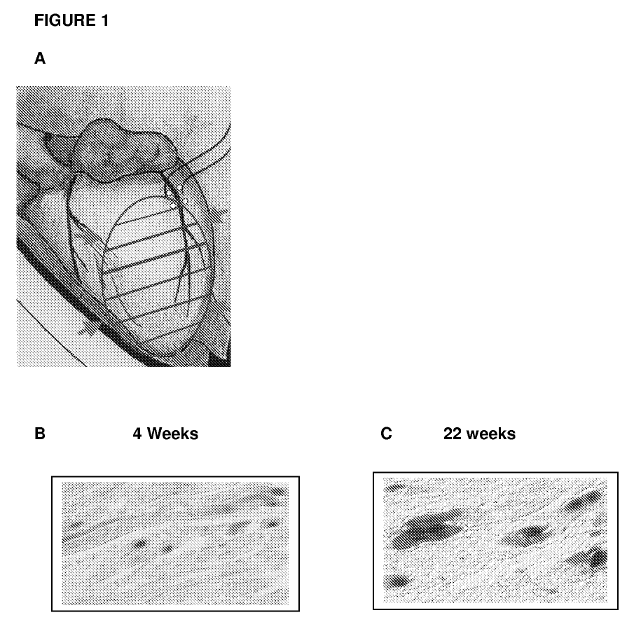

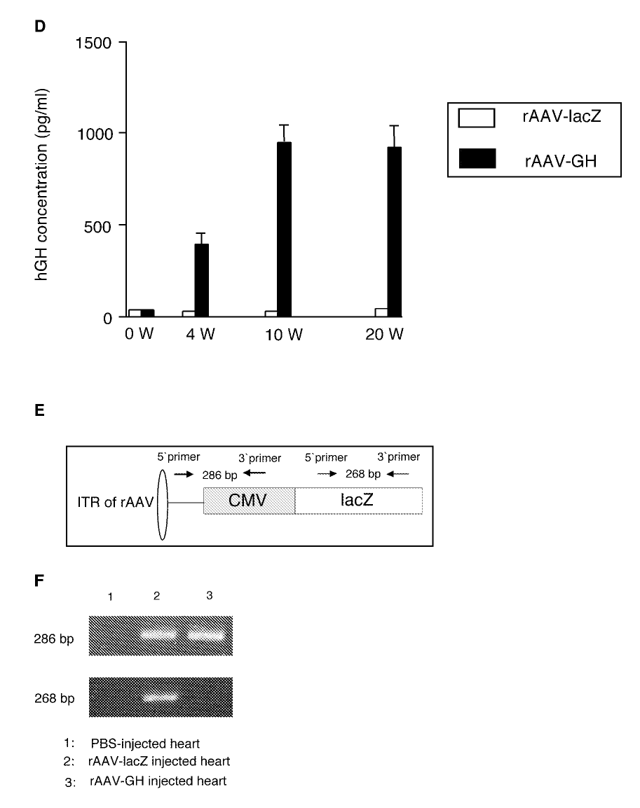

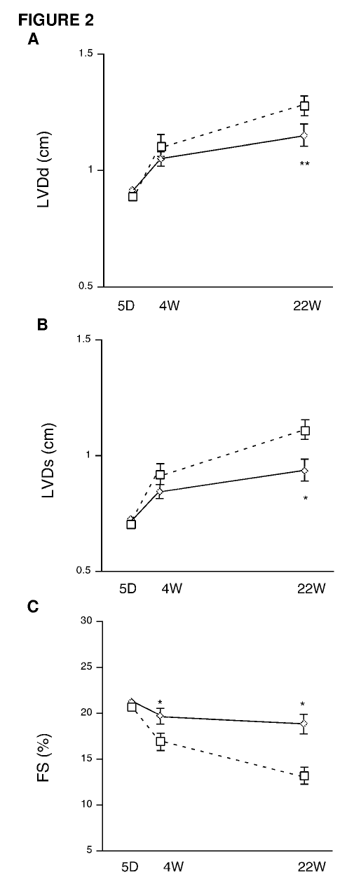

Also, as is demonstrated herein, local delivery of the human growth hormone (hGH) gene by rAAV vector significantly improved cardiac function and ventricular remodeling following myocardial infarction. Using echocardiography, it was observed that GH expression significantly improved % FS not only in acute phase but also in chronic phase following myocardial infarction (MI) (FIG. 2C). Recombinant AAV vector-mediated GH expression lasted up to 22 weeks following a single infection (FIG. 1C-E). This is the first time demonstrating the presence of the rAAV genome as well as a sustained GH expression 22 weeks after infection. Sustained effects of rAAV-mediated GH expression on angiogenesis, cell proliferation and apoptosis after MI were observed. In this study, rAAV was administered after open chest surgery. Clinical methods for delivering rAAV-GH include direct cardiac injection by coronary artery catheter, direct muscle injection using the NOGA mapping system (Losordo et al., Circulation, 98:2800-2804, 1998), or by any other means designed for direct or indirect cardiac administration (see, e.g., U.S. Pat. No. 6,723,082). The invention is not limited by the exact means of delivery of the rAAV to the subject. During stable GH expression, it was found that the maximum concentration of human GH was 1.3 ng/ml in the serum of rAAV-GH infected rats (FIG. 1D). This level is much lower than the normal GH level for adult rats (2-10 pg/ml). In addition, although echocardiography demonstrated that GH expression mildly induced cardiac hypertrophy, no tumor genesis or local hump of heart muscle tissue in rAAV-infected portions were observed.

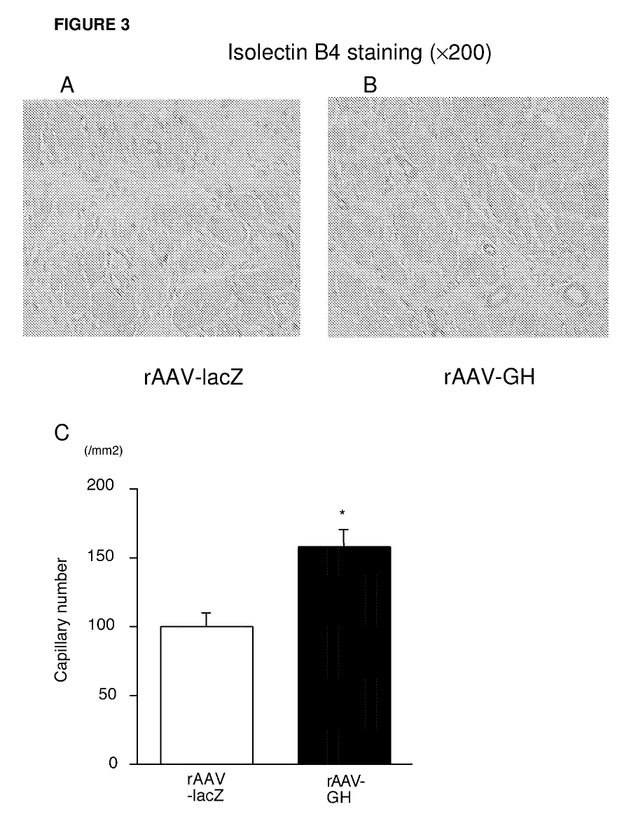

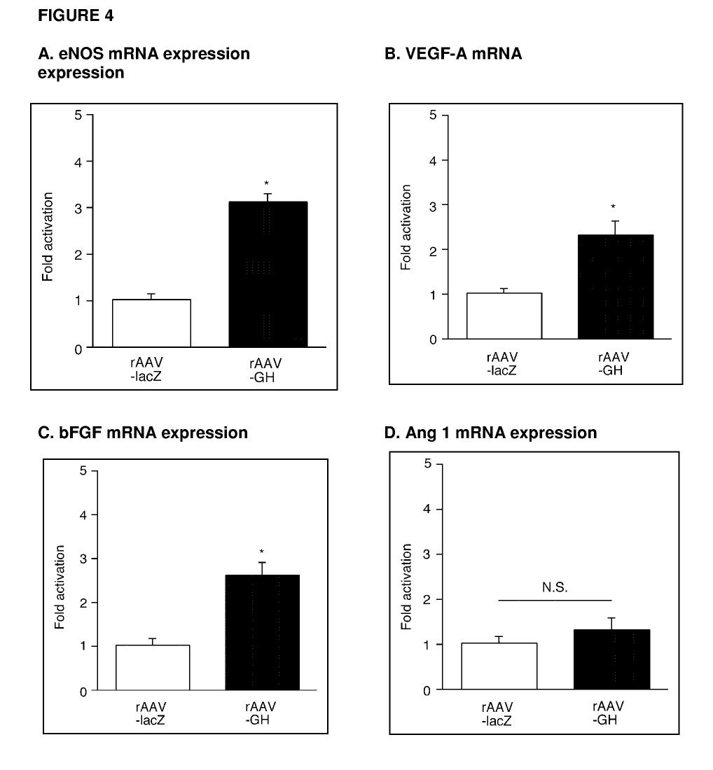

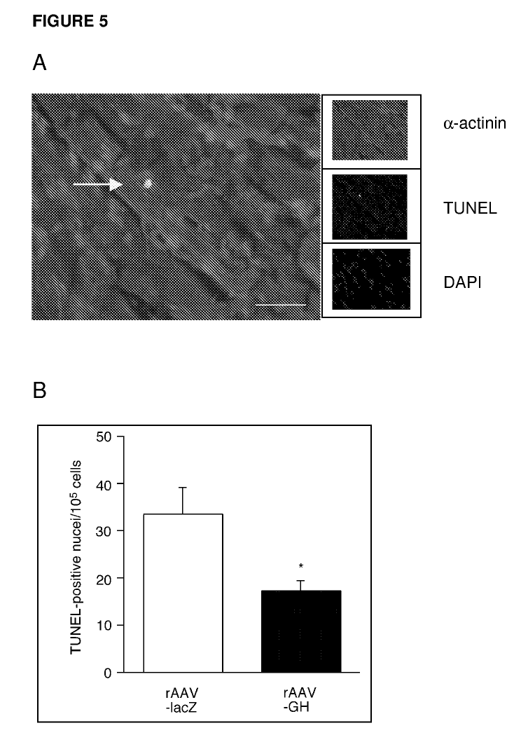

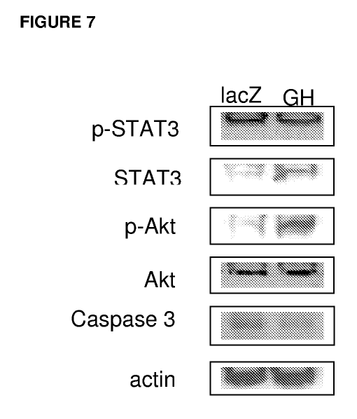

It is demonstrated herein that GH expression using a rAAV-GH viral vector induced angiogenesis in the heart compared to the control lacZ group (FIG. 3A-3C). Expression of angiogenic factors eNOS, VEGF and bFGF gene expression were significantly increased after rAAV-GH vector infection (FIG. 4A-D) suggesting that these critical angiogenic factors play an important role in GH-induced capillary formation. Moreover, overexpression of growth hormone prevented cardiomyocyte apoptosis from ischemia (FIG. 5A-5B). In rAAV-GH vector infected hearts, significant activation of Akt and proliferating cell nuclear antigen (PCNA); and down-regulation of caspase 3 in the peri-infact area of heart muscle was observed (FIG. 7).

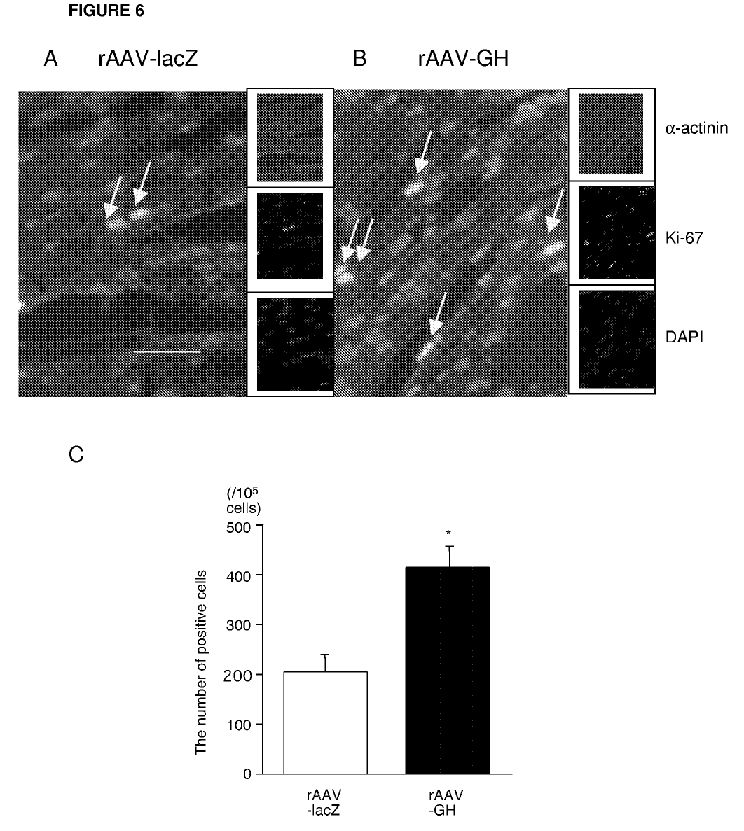

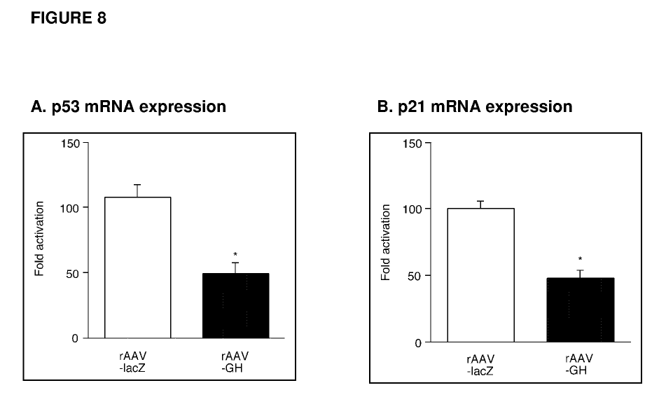

GH significantly induced proliferating cell nuclear antigen (PCNA) expression and increased the number of Ki-67 positive cardiac myocytes (FIG. 6A-C) and down-regulated mRNA expression of cell cycle inhibitory proteins, p53 and p21 (FIGS. 8A and B). These results indicate that GH promotes cardiomyocyte proliferation.

It is demonstrated herein that GH expression via rAAV improves cardiac function in the chronic stage post-MI (FIG. 2A-C). Collectively, these data demonstrate that local delivery of GH gene by rAAV can provide an effective approach for the prevention and/or treatment of various cardiomyopathies.

The invention features compositions and methods that are useful for treating or preventing a cardiac disease in a subject. Such compositions and methods are particularly useful for increasing angiogenesis, increasing cell proliferation, and reducing apoptosis in a cardiac tissue following a myocardial infarction. The invention is based, in part, on the discovery that expression of growth hormone in cardiac muscle following a myocardial infarction increases angiogenesis, reduces apoptosis, and increases cardiac function.

The present invention provides methods of preventing or treating cardiac diseases and/or disorders or symptoms thereof which comprise administering a therapeutically effective amount of a pharmaceutical composition comprising an expression vector (e.g., recombinant adeno-associated viral vector) comprising a nucleotide sequence for the expression of growth hormone polypeptide, fragment thereof, or mimetic, of the formulae herein to a subject (e.g., a mammal such as a human). Thus, one embodiment is a method of treating a subject suffering from or susceptible to a cardiac disease or disorder or symptom thereof. The method includes the step of administering to the mammal a therapeutic amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition. Prevention or prophylactic treatment can require administration of more than one dose of the compositions of the invention.

The therapeutic methods of the invention (which include prophylactic treatment) in general comprise administration of a therapeutically effective amount of the compounds herein, such as a compound of the formulae herein to a subject in need thereof, including a mammal, particularly a human. Such treatment will be suitably administered to subjects suffering from, having, susceptible to, or at risk for a disease, disorder, or symptom thereof. Therapeutic methods can require administration of more than one dose of the compositions of the invention. Determination of those subjects “at risk” can be made by any objective or subjective determination by a diagnostic test or opinion of a subject or health care provider (e.g., genetic test, enzyme or protein marker, functional test, Marker (as defined herein), family history, and the like). The compounds herein may be also used in the treatment of any other disorders in which apoptosis in a cardiac muscle may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay, functional assay) in a subject suffering from or susceptible to a disorder or symptoms thereof associated with the apoptosis of a cardiac cell or with a myocardial infarction, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment. Periodic diagnostic measurements can be similarly made and compared to determine the efficacy of therapeutic interventions.

### Prophylactic and Therapeutic Methods

The invention provides method for preventing, treating or reducing the severity of a cardiac disease. Exemplary cardiac diseases include, but are not limited to, myocardial infarction, cardiac hypertrophy, reduced systolic function, reduced diastolic function, maladaptive hypertrophy, heart failure with preserved systolic function, diastolic heart failure, hypertensive heart disease, aortic and mitral valve disease, pulmonary valve disease, hypertrophic cardiomyopathy, post ischemic and post-infarction cardiac remodeling and cardiac failure. Methods of the invention are particularly suitable for use in cardiac diseases directly or indirectly associated with ischemia (myocardial ischemia), an infarct (myocardial infarction), congestive heart failure (CHF) and related heart muscle disorders, such as cardiomyopathy and cardiomyositis. Desirably, methods of the invention are also used to prevent, treat, or reverse the pathological effects of a cardiac disease by increasing cardiac function, angiogenesis, cell proliferation in a cardiac muscle, while decreasing apoptosis in the heart of a subject having or having a propensity to develop a cardiac disease.

To determine a subject's propensity to develop a cardiac condition, the subject's cardiac risk is assessed using any standard method known in the art. Important indicators of cardiac risk are age, hereditary factors, weight, smoking, blood pressure, exercise history, and diabetes. Other indicators of cardiac risk include the subject's lipid profile, which is typically assayed using a blood test, or any other biomarker associated with heart disease or hypertension. Other methods for assaying cardiac risk include, but are not limited to, an EKG stress test, thallium stress test, EKG, CT scan, echocardiogram, magnetic resonance imaging study, non-invasive and invasive arteriogram, and cardiac catheterization.

### Cardiovascular Function

Compositions of the invention may be used to enhance cardiac function in a subject having reduced cardiac function. Desirably, cardiac function is increased by at least 5%, 10% or 20%, or even by as much as 25%, 50% or 75%. Most advantageously, cardiac function is enhanced or damage is reversed, such that the function is substantially normal (e.g., 85%, 90%, 95%, or 100% of the cardiac function of a healthy control subject). Alternatively, such assays are used to monitor the condition of a subject prior to, during, or following treatment with an expression vector of the invention. Treatments that increase cardiac function are useful in the methods of the invention.

Any number of standard methods are available for assaying cardiovascular function. Preferably, cardiovascular function in a subject (e.g., a human) is assessed using non-invasive means, such as measuring net cardiac ejection (ejection fraction, fractional shortening, and ventricular end-systolic volume) by an imaging method such echocardiography (see, e.g., FIG. 2), nuclear or radiocontrast ventriculography, or magnetic resonance imaging, and systolic tissue velocity as measured by tissue Doppler imaging. Systolic contractility can also be measured non-invasively using blood pressure measurements combined with assessment of heart outflow (to assess power), or with volumes (to assess peak muscle stiffening). Measures of cardiovascular diastolic function include ventricular compliance, which is typically measured by the simultaneous measurement of pressure and volume, early diastolic left ventricular filling rate and relaxation rate (can be assessed from echoDoppler measurements). Other measures of cardiac function include myocardial contractility, resting stroke volume, resting heart rate, resting cardiac index (cardiac output per unit of time [L/minute], measured while seated and divided by body surface area [m²])) total aerobic capacity, cardiovascular performance during exercise, peak exercise capacity, peak oxygen (O₂) consumption, or by any other method known in the art or described herein. Measures of vascular function include determination of total ventricular afterload, which depends on a number of factors, including peripheral vascular resistance, aortic impedance, arterial compliance, wave reflections, and aortic pulse wave velocity. The method of monitoring cardiovascular function is not a limitation of the invention.

### Polynucleotide Therapy

Polynucleotide therapy featuring a polynucleotide encoding a growth hormone or variant, or fragment thereof is one therapeutic approach for treating a cardiac disease. Such nucleic acid molecules can be delivered to cells of a subject having a cardiac disease. The nucleic acid molecules must be delivered to the cells of a subject (e.g., cardiac cells) in a form in which they can be taken up so that therapeutically effective levels of a human growth hormone (e.g., a human growth hormone, a human growth hormone variant) or fragment thereof can be produced. Desirably, expression of a therapeutic gene in a cell, such as a cardiac cell, can occur for at least 2, 3, 4, 5, 6, 7, 8, 12, 16, 18, 20, 22, or 24 weeks in vivo after administration of the cell to a host subject, or for longer periods.

Transducing viral (e.g., retroviral, adenoviral, and adeno-associated viral) vectors can be used for somatic cell gene therapy, especially because of their high efficiency of infection and stable integration and expression. In a preferred embodiment, the viral vector is a rAAV vector. For example, a polynucleotide encoding a human growth hormone, variant, or a fragment thereof, can be cloned into a rAAV vector and expression can be driven from an endogenous rAAV promoter, or from a promoter specific for a target cell type of interest. In one embodiment, a viral vector is used to administer growth hormone polynucleotide systemically. In an alternative embodiment, the viral vector is delivered to the heart. In another embodiment, the viral vector can be delivered both systemically and directly to the heart, concurrently or sequentially.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant growth hormone protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### Expression Vectors

Typically a mammalian expression vector utilizes a promoter adjacent to a transgene to express the corresponding mRNA that can be translated to the corresponding protein or polypeptide in the cell. As used herein, a “promoter” refers to a DNA sequence to which RNA polymerase binds to initiate transcription of messenger RNA, and to which other regulatory elements bind to facilitate, regulate, enhance or suppress transcription. A promoter that is “operably linked” to a DNA sequence encoding a gene or a fragment thereof in a vector causes the DNA sequence to be expressed or produced when the vector is introduced into a cell or is provided with suitable substrates and conditions in vitro. The promoter of the invention can be a “ubiquitous” promoter active in essentially all cells of a host organism (such as a human), for example, a CMV, beta-actin or optomegalovirus promoters, or it may be a promoter whose expression is more or less specific to the target cell or tissue. An example of a useful promoter which could be used to express a gene of interest according to the invention is a cytomegalovirus (CMV) immediate early promoter (CMV IE) (Xu et al., Gene 272: 149-156, 2001). These promoters confer high levels of expression in most animal tissues, and are generally not dependent on the particular encoded proteins to be expressed. Examples of other such promoters of use in the invention include Rous sarcoma virus promoter, adenovirus major late promoter (MLP), Herpes Simplex Virus promoter, HIV long terminal repeat (LTR) promoter, beta actin promoter (Genbank Accession No. K00790), or murine metallothionein promoter (Stratagene San Diego Calif.). Examples of tissue- or cell-specific promoters are described infra. The latter type of promoters can be used to advantage, for example to restrict expression of transgenes to cells having tropism for particular serotypes of rAAV.

As discussed, transfection refers to a process of delivering heterologous DNA, such as a viral vector encoding a transgene of interest, or plasmid DNA to a cell by physical or chemical methods. The DNA is transferred into the cell by any suitable means, such as electroporation, calcium phosphate precipitation, or other methods well known in the art. Use of the term “transduction” encompasses both introducing the gene or gene cassette into a cell for purposes of tracking (as with a reporter gene), or for delivering a therapeutic gene or correcting a gene defect in a cell. Transduction in the context of producing viral vectors for gene therapy (for example rAAV vectors) in a cell can also mean introduction of a gene or gene cassette into a producer cell to enable the cell to produce rAAV. The rAAV particles made by the producer cells are subsequently purified by standard methods known in the art and as described below.

As discussed above, typical transgenes comprise a heterologous gene sequence, or a recombinant construct of multiple genes (“gene cassette”) in a vector. The recombinant AAV vectors of the invention can be produced in vitro by introducing gene constructs into cells known as producer cells. The term “producer cell” refers one of many known cell lines useful for production of rAAV, into which heterologous genes are typically introduced by viral infection or transfection with plasmid DNA. As used herein, the term “infection” refers to delivery of heterologous DNA into a cell by a virus. Infection of a producer cell with two (or more) viruses at different times is referred to as “co-infection.”

In general, systems for producing rAAV comprise three basic elements: 1) a gene cassette containing one or more genes of interest, 2) a gene cassette containing AAV rep and cap genes and 3) a source of “helper” virus proteins.

Typically the first gene cassette is constructed with the gene of interest flanked by inverted terminal repeats (ITRs) from AAV. Particular genes of interest of use in the invention have been described supra. A suitable vector for expressing one or more reporter genes is pAAV-CMV-lacZ. This vector comprises a CMV promoter and drives expression of the lacZ gene. For more restricted expression of transgenes, other suitable vectors are constructed with cell-specific promoters, such as the vector described in which restricts expression of the transgene to cardiac muscle cells. Other suitable promoters are described infra.

As discussed, preferred transgenic cells of the invention are stably transduced with the rAAV vectors. Within the rAAV system, ITRs function to direct integration of the gene of interest into the host cell genome, thereby facilitating stable transduction (Hermonat and Muzyczka, Proc Natl Acad Sci USA. 81(20):6466-70, 1984; Samulski, et al., Cell. 33(1):135-43. 1983).

The second gene cassette contains rep and cap, AAV genes encoding proteins needed for replication and packaging of rAAV. The rep gene encodes four proteins (Rep 78, 68, 52 and 40) required for DNA replication. The cap genes encode three structural proteins (VP1, VP2, and VP3) that make up the virus capsid.

The third element is required because AAV-2 does not replicate on its own. “Helper functions” are protein products from helper DNA viruses that create a cellular environment conducive to efficient replication and packaging of rAAV. Adenovirus (Ad) has been used extensively to provide helper functions for rAAV. The gene products provided by Ad are encoded by the genes E1a, E1b, E2a, E4 or E6, and Va (Hauswirth et al., Methods Enzymol. 316:743-61, 2000).

The rAAV vectors used can be produced in vitro, using suitable producer cell lines, such as 293 (ATCC No. CRL-1573) and HeLa (ATCC No. CCL-2). Alternatively in some instances the rAAV vectors can be purchased from commercial sources. A well-known strategy for delivering all of the required elements for rAAV production utilizes two plasmids and a helper virus. This method relies on transfection of the producer cells with plasmids containing gene cassettes encoding the necessary gene products, as well as infection of the cells with Ad to provide the helper functions. This system employs plasmids with two different gene cassettes. The first is a proviral plasmid encoding the recombinant DNA to be packaged as rAAV. The second is a plasmid encoding the rep and cap genes. To introduce these various elements into the cells, the cells are infected with Ad as well as transfected with the two plasmids. Alternatively, in more recent protocols, the Ad infection step can be replaced by transfection with an adenovirus “helper plasmid” containing the VA, E2A and E4 genes (Xiao, et al., J. Virol. 72(3):2224-32. 1998, Matsushita, et al., Gene Ther. 5(7):938-45.1998).

While Ad has been used conventionally as the helper virus for rAAV production, other DNA viruses, such as Herpes simplex virus type 1 (HSV-1) can be used as well. The minimal set of HSV-1 genes required for AAV-2 replication and packaging has been identified, and includes the early genes UL5, UL8, UL52, and UL29 (Muzyczka and Burns, supra). These genes encode components of the HSV-1 core replication machinery, i.e., the helicase, primase, primase accessory proteins, and the single-stranded DNA binding protein (Knipe, Adv Virus Res. 37:85-123, 1989; Weller, J Gen Virol. 71 (Pt 12):2941-52 1991). This rAAV helper property of HSV-1 has been utilized in the design and construction of a recombinant Herpes virus vector capable of providing helper virus gene products needed for rAAV production (Conway et al., Gene Ther. 6(6):986-93, 1999).

A preferred method for preparing the rAAV vectors of the invention is described, for example, in Snyder et al., 1997 (Nat. Genet. 8:270-276). Briefly, subconfluent 293 cells are co-transfected with vector plasmid and pLTAAVhelp using calcium phosphate. Cells are then infected with adenovirus Ad5dl312 (an E1A-deletion mutant) at a multiplicity of infection of about 2. The E1A-deleted rAd-lacZ vector can be prepared for example as described in Hardy et al., 1997 (J. Virol. 71:1842-1849). After approximately 72 hours, the cells are harvested and lysed by repeated (for example, three) freeze/thaw cycles. Ad is heat-inactivated, and the rAAV virions are purified, for example on cesium chloride gradients. The gradient fractions containing rAAV are dialyzed against sterile PBS, and stored at about −80° C. Particle titers (preferably of about 1˜2×10¹²/ml) can be determined, for example, by dot blot analysis.

Recombinant AAV vectors have generally been based on AAV-2 capsids. It has recently been shown that rAAV vectors based on capsids from AAV-1, AAV-3, or AAV-4 serotypes differ substantially from AAV-2 in their tropism. Capsids from other AAV serotypes offer advantages in certain in vivo applications over rAAV vectors based on the AAV-2 capsid. For example, rAAV vectors with particular serotypes may increase the efficiency of gene delivery and integration into the genome of certain types of cells. Although it is shown in Examples below that rAAV-2 is an effective vector serotype for transduction and stable integration into cardiac cells, the invention is not so limited. Further, it may be advantageous to have available alternative rAAV vectors based on multiple AAV serotypes. For example, this could become important if re-administration of rAAV vector becomes clinically necessary. This can be achieved by administering a rAAV particle whose capsid is composed of proteins from a different AAV serotype, not affected by the presence of a neutralizing antibody to the first rAAV vector (Xiao, et al., supra). For the above reasons, recombinant AAV vectors constructed using cap genes from serotypes other than, or in addition to AAV-2, are desirable.

In some circumstances the promiscuous tropism of rAAV may lead to the undesirable expression of therapeutic genes in non-targeted cells. This limitation may be overcome by the use of tissue-specific promoters. Liver-, brain-, cancer-, and rod photoreceptor-specific expression can be achieved, for example, using tissue-specific promoters, such as those from albumin, enolase, calcitonin, and rodhopsin, respectively. Muscle-specific expression in skeletal muscle can be directed, for example, by a rAAV vector comprising a muscle creatine kinase (MCK) promoter.

For cardiac-specific expression, a suitable promoter is an alpha myosin heavy chain (MHC) gene promoter, myosin light chain (MLC) promoter 2v (MLC-2v). rAAV vectors expressing a therapeutic or reporter gene under the control of a cardiac-specific promoter can be made, for example, as described in Aikawa et al, 2002 (supra) by cloning fragments of the α-MHC promoter (−344 to +19), a larger promoter fragment containing the PNR (−344 to +119), or the α-MHC enhancer (−344 to −156) together with a heterologous promoter to control transgene expression. Long-term cardiac expression of both therapeutic and reporter genes with low cytotoxicity can be attained using these constructs.

### Pharmaceutical Compositions

For therapeutic uses, the compositions or agents identified using the methods disclosed herein can be administered directly to a desired tissue (e.g., a cardiac tissue) or systemically, for example, formulated in a pharmaceutically-acceptable buffer such as physiological saline. Preferable routes of administration include, for example, subcutaneous, intravenous, interperitoneally, intramuscular, intracardiac, or intradermal injections that provide continuous, sustained levels of growth hormone in the patient. Treatment of human patients or other animals will be carried out using a therapeutically effective amount of an expression vector encoding a therapeutic polypeptide in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the cardiac disease. Generally, amounts will be in the range of those used for other viral agents used in the transduction of a target tissue or for the treatment of other diseases associated with a cardiac disease. In certain instances lower amounts will be needed because of the increased specificity of the compound, for example due to promoter selection or site of administration. An expression vector of the invention is administered at a dosage that controls the clinical or physiological symptoms of a cardiac disease as determined by a diagnostic method known to one skilled in the art. In one embodiment, a pharmaceutical composition comprise a replication defective rAAV vector that encodes a therapeutic polypeptide. The serotype of the rAAV vector can be any suitable serotype, such as AAV-1, AAV-2, or another available serotype. Examples include AAV-3, AAV-4, AAV-5, AAV-6, AAV-7, AAV-8, AAV-9, or AAV-10.

The invention further provides a simple means for identifying compositions (including nucleic acids, peptides, small molecule inhibitors, and mimetics) capable of acting as therapeutics for the treatment of a cardiac disease or symptom thereof.

### Methods of Delivery

In particular applications involving cardiac and cardiovascular disorders, the cell or vector of the invention can be administered directly to the heart. Desirably, such methods are sufficient to transducer at least one cardiac cell in vivo. The transduced cell expresses a transgene (e.g., therapeutic polypeptide, such as human growth hormone) for at least 1, 2, 3, 4, 5, 6, 7, or 8 weeks after administration of the vector of the invention to the subject. In some embodiments, expression can continue for three, six, nine, twelve months or even longer following administration to a subject.

### Screening Assays

As described in more detail below, expression of human growth hormone in a cardiac tissue decreases apoptosis, increases angiogenesis, and increases cardiac cell proliferation following a myocardial infarction. Based in part on this discovery, compositions of the invention are useful for the high-throughput low-cost screening of candidate compounds, such as polypeptides, fragments thereof, polypeptide analogs that have similar effects. A fragment is a portion of a polypeptide or nucleic acid molecule that is of a length sufficient to have at least one biological activity attributed to the polypeptide or nucleic acid molecule from which the fragment is derived. Exemplary biological activities of a therapeutic polypeptide include reducing apoptosis, increasing angiogenesis, or increasing proliferation of a cell of interest.

Assays for measuring cell apoptosis are known to the skilled artisan. Apoptotic cells are characterized by characteristic morphological changes, including chromatin condensation, cell shrinkage and membrane bleeding, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

Methods for measuring an increase in angiogenesis are also known in the art and are described herein. In general, angiogenesis can be assayed by measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area).

Methods of assaying cell growth and proliferation are known in the art. See, for example, Kittler et al. (Nature. 432 (7020): 1036-40, 2004) and Miyamoto et al. (Nature 416(6883):865-9, 2002). Assays for cell proliferation generally involve the measurement of DNA synthesis during cell replication. In one embodiment, DNA synthesis is detected using labeled DNA precursors, such as ([³H]-Thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

Assays for measuring cell survival are known in the art, and are described, for example, by Crouch et al. (J. Immunol. Meth. 160, 81-8); Kangas et al. (Med. Bio. 1.62, 338-43, 1984); Lundin et al., (Meth. Enzymo1.133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett.1:611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

In some embodiments, a therapeutic polypeptide is provided together with a second compound that promotes angiogenesis, reduces apoptosis, or increases cell proliferation. Such factors include VEGF, particularly VEGF-1, VEGF165, and certain cell matrix proteins, such as fibronectin. The second compound can be delivered as a peptide or in a second rAAV.

### Compounds Suitable for Use in Methods of the Invention

The invention provides expression vectors comprising a nucleic acid sequence encoding growth hormone to increase angiogenesis, reduce apoptosis, or increase cell proliferation a cardiac tissue. The amino acid sequence of human growth hormone is provided, for example, at GenBank Accession No. P01241 and in the sequence listing as SEQ ID NO: 1. The sequence of a nucleic acid molecule encoding a human growth hormone is provided at GenBank Accession No. BC075013 (SEQ ID NO: 2). Accession numbers for growth hormone from other species are provided above and many others are available for example through the NCBI-BLAST database. Expression of the therapeutic gene in a transgenic cardiac cell can occur for at least 2, 3, 4, 5, 6, 7, 8, 12, 16, 18, 20, 22, or 24 weeks in vivo after administration of the cell to a host subject, or for longer periods.

### Combination Therapies

In some embodiments a vector of the invention is administered together with other therapeutics used for the treatment of a cardiac disease, or used to increase angiogenesis, increase proliferation, or reduce apoptosis. If desired, the vector is administered together with an angiogenic factor. An “angiogenic factor” is any polypeptide or functional fragment thereof that increases, supports or promotes angiogenesis. In one version of the method, at least one nucleic acid encoding at least one angiogenic factor or a functional fragment thereof is administered to the subject in combination with a vector expressing growth hormone (GH).

Angiogenic factors and mitogens include acidic and basic fibroblast growth factors (aFGF and bFGF), vascular endothelial growth factor (VEGF-1), VEGF165, epidermal growth factor (EGF), transforming growth factor α and β (TGF-α and TFG-β), platelet-derived endothelial growth factor (PD-ECGF), platelet-derived growth factor (PDGF), tumor necrosis factor α (TNF-α), hepatocyte growth factor (HGF), insulin like growth factor (IGF), erythropoietin, colony stimulating factor (CSF), macrophage-CSF (M-CSF), granulocyte/macrophage CSF (GM-CSF), angiopoetin-1 (Ang1) and nitric oxide synthase (NOS); and functional fragments thereof. Muteins or functional fragments of a mitogen may be used as long as they have at least some of the desirable properties of the parent compound.

### Kits

The invention provides kits for the treatment or prevention of a cardiac disease associated with cardiac ischemia. In one embodiment, the kit includes a pharmaceutical pack comprising an effective amount of a recombinant adeno-associated viral vector comprising a growth hormone encoding polynucleotide sequence. Preferably, the compositions are present in unit dosage form. In some embodiments, the kit comprises a sterile container that contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention or combinations thereof are provided together with instructions for administering them to a subject having or at risk of developing a cardiac disease associated with ischemia. The instructions will generally include information about the use of the compounds for the treatment or prevention of a cardiac disease associated with ischemia. In other embodiments, the instructions include at least one of the following: description of the compound or combination of compounds; dosage schedule and administration for treatment of a cardiac disease associated with ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The following examples are provided to illustrate the invention, not to limit it. Those skilled in the art will understand that the specific constructions provided below may be changed in numerous ways, consistent with the above described invention while retaining the critical properties of the compounds or combinations thereof.

### EXAMPLES

### Example 1

### rAAV Vector Comprising Human Growth Hormone

Standard serotype 2 rAAV vectors were produced essentially as described Aikawa et al. (J. Biol. Chem., 277:18979-18985, 2002) using standard methods. Briefly, a nucleic acid sequence encoding human growth hormone was amplified using primers that included sequences to add an EcoR1 site onto one end of the human growth hormone coding sequence, and a BamH1 site onto the other. (The nucleic acid sequence encoding human growth hormone is provided at Genbank Accession No. BC075013 (SEQ ID NO: 2).) The amplification product was digested using the appropriate restriction enzymes and inserted into the vector plasmid at the corresponding sites (SEQ ID NO: 4). Each vector plasmid was cotransfected into subconfluent 293 cells with the pLTAAVhelp helper plasmid using the calcium phosphate method.

Cells were then infected with adenovirus Ad5dl312 (an E1A-null mutant) at a multiplicity of infection of 2. After 72 hours the cells were harvested, lysed by three freeze/thaw cycles, and the virions were isolated by cesium chloride gradient centrifugation. The gradient fractions containing rAAV were dialyzed against sterile PBS, heated for 30 minutes at 56° C., and stored at −80° C. The particle titer was determined by quantitative real-time PCR and typically contains about 5×10¹² particles/ml.

### Example 2

### Induction of myocardial infarction and administration of hGH-rAAV

All procedures were performed in accordance with Caritas St. Elizabeth's Institutional Animal Care and Use Committee. 7-8 week-male Sprague-Dawley rats (Jackson Laboratory, Bar Harbor, Me.) were used. They were anesthetized with an intraperitoneal injection of ketamine (40-90 mg/kg) and xylazine (5-10 mg/kg) and the respiration of the anesthetized rat was controlled using an animal ventilator for a thoracotomy incision. Myocardial infarction (MI) was induced by ligating the proximal left anterior descending coronary artery with 6-0 prolene suture. Following induction of myocardial infarction, either the rAAV-lacZ vector or the rAAV-hGH vector was directly injected with 1×10¹¹ particles in 20 μl volume using a 30-gauge needle to 5 sites (total 5×10¹¹ particles) within the myocardium around the infarcted area (FIG. 1A). The post-operative survival rate of this operation was more than 90%. Observations were made for up to 22 weeks post-MI/viral injection.

### Example 3

### Assays for Expression of β-Galactosidase and Hgh

Four weeks after infection of rAAV vectors, the heart was harvested. For detection of β-galactosidase activity, freshly excised tissues in O.C.T. compound (Sakura), were flash frozen and sectioned. After fixation, slides were stained overnight with 5-bromo-4-chloro-3-iodolyl-beta-D-galactopyranoside (X-gal) using routine methods.

The ability of rAAV to transduce rat heart muscle post-MI was confirmed using a rAAV-lacZ vector. The left anterior descending coronary artery was ligated to induce myocardial infarction, and a total of 5×10¹¹ rAAV vectors were delivered by direct injection to five different sites within the peri-infarct area (see, FIG. 1A). Four weeks after infection, the heart was harvested and rAAV-lacZ mediated transduction was assayed for β-galactosidase activity in the myocardium. The P-galactosidase expression was prominently observed along the infarct area (FIGS. 1B and 1C).

To develop a potential human growth hormone (GH) therapy for MI with sustained expression of GH, 5×10¹¹ particles of rAAV-GH were directly injected into the myocardium as above, post-MI. Periodically, after injection with rAAV-LacZ or rAAV-GH, blood samples were taken from the tail vein of anesthetized rats, and the plasma hGH concentrations were determined by the Roche hGH ELISA assay kit. Only background levels of hGH were detected in rAAV-lacZ control group; however, circulating hGH levels were significantly increased from 4 weeks and continued to 22 weeks after rAAV-GH injection (FIG. 1D). These results demonstrate that hGH is expressed by rAAV vectors in cardiac cells post-MI for a sustained period.

To further verify the gene transfer following injection of rAAV vectors, total DNA was isolated from the heart using the Puregene® DNA isolation kit (Gentra), and the presence of rAAV genome was analyzed by PCR. A schematic of the constructs and the PCR primers used are shown in FIG. 1E. In rAAV-lacZ vector-transduced tissues 22 weeks after infection, agarose gel electrophoresis demonstrated both a 286-bp band corresponding to the inverted terminal repeat (ITR) sequence of rAAV genome to CMV promoter sequence and a 268-bp band corresponding to lacZ gene (FIG. 1F, lane 2). In hearts transduced with rAAV-GH vector, only the 286-bp PCR product was observed (FIG. 1F, lane 3). No product was seen in from PBS-injected (control) samples. These data demonstrate the presence of intact rAAV DNA at least 22 weeks post injection of the rAAV vectors.

### Example 4

### Assay for Modulation of Cardiac Function Post-MI/rAAV Injection

Transthoracic echocardiography (SONOS 5500, PHILIPS) was performed at day 5 and 22 weeks after myocardial infarction with rAAV infection. Left ventricular (LV) diastolic dimension (LVDd), systolic dimension (LVDs) and fractional shortening (FS) were measured at the midpapillary muscle level. All measurements were examined by an expert researcher who was blinded to the treatment group.

Transthoracic echocardiography showed that at baseline, left ventricular diastolic dimension (LVDd), left ventricular systolic dimension (LVDs) and fractional shortening (FS) were similar between the rAAV-lacZ control group and the rAAV-GH treated group (FIG. 2A-C). Four weeks after infarction, there was a significant difference in % FS between the two groups. Furthermore, in both groups, LVDd and LVDs were significantly increased and FS conversely was decreased 22 weeks after myocardial infarction. However, LVDd and LVDs were significantly lower in the rAAV-GH group (1.15±0.05 and 0.93±0.04 cm) compared to the rAAV-lacZ group (1.27±0.04 and 1.10±0.04 cm). FS of the rAAV-GH group by 22 weeks (19.0±1.1%) was significantly higher compared to the control group (13.0±0.8%). In addition, echocardiography showed hypertrophy of the posterior wall in the GH group compared to the control group (0.15±0.02 vs 0.12±0.02 cm). These results indicate that GH expression by rAAV improved cardiac function and the remodeling post-MI in rats.

### Example 5

### Assay for Angiogenesis in Heart Post-MI/rAAV Injection

Twenty-two weeks after myocardial infarction, tissue samples were harvested, fixed with 4% paraformaldehyde (PFA), and immunohistochemically stained using antibodies prepared against a rat specific endothelial cell marker, isolectin B4 (Vector Laboratories) (FIGS. 3A and B). Capillary density was evaluated morphometrically by histological examination of 5 randomly selected fields of tissue sections of peri-infarct LV myocardium. Capillaries were recognized as tubular structures positive for isolectin B4.

Immunohistochemical analysis revealed that capillary density in the heart of the rAAV-GH group was significantly higher than that of the rAAV-lacZ control group (98.75±9.74 versus 156.25±11.5; FIG. 3A-3C) 22 weeks after infection. These data demonstrate that administration of rAAV-GH post-MI promotes angiogenesis.

### Example 6

### Assay for Angiogenic Factors in Heart Post-MI/rAAV Injection

Neovessels form in response to stimulation by soluble angiogenic factors that regulate endothelial migration, proliferation, and survival. The best studied factors described to date, vascular endothelial growth factor (VEGF), basic fibroblast growth factor (bFGF) and the angiopoietin-1 (Ang 1), have emerged as regulators of the angiogenic process. In addition, endothelial nitric oxide synthase (eNOS) is known as a downstream target for VEGF-induced angiogenesis. To confirm expression of mRNAs involved in angiogenesis, quantitative RT-PCR was performed 4 weeks after infarction and viral vector injection.

Total RNA was extracted from heart tissue with RNA-Stat (Tel-Test) according to the manufaturer's instructions. First-strand cDNA was generated using the Taqman Multiscribe Reverse Transcription Kit (Applied Biosystems) primed with a mix of oligo dT and Random Hexamers. Gene expression was determined by Taqman real-time quantitative PCR on the 7300 Sequence Detection System (Applied Biosystems) using Taqman PCR Master Mix (Applied Biosystems). Taqman primer/probe sets (Biosearch Technologies) were designed using the Primer Express Software (Applied Biosystems). PCR Conditions were as follows: hold for 2 minutes at 50° C. and 10 minutes at 95° C. followed by 2 step PCR for 40 cycles of 95° C. for 15 seconds and 60° C. for 60 seconds with fluorescence monitoring at the end of each elongation step. Relative mRNA expression of target genes was calculated with the comparative threshold cycle (CT) method. All target sequences were normalized to GAPDH in multiplexed reactions performed in duplicate. Differences in CT values were calculated for each target mRNA by subtracting the mean value of GAPDH.

Although there was no significant difference in Ang 1 expression between rAAV-lacZ group and rAAV-GH group, overexpression of growth hormone from the rAAV vector significantly increased gene expression of eNOS, VEGF and bFGF more than two fold compared to control (FIG. 4A-D). In addition, an increase in eNOS mRNA expression in animals treated with rAAV-GH 22 weeks after infection was observed. These data demonstrate the induction of expression of multiple angiogenic related proteins by rAAV-GH.

### Example 7

### Assay for Apoptosis and Proliferation in Heart Post-MI/rAAV Injection

Twenty-two weeks after infarction, cardiac tissue was harvested, and triple staining with α-actinin for cardiomyocytes, TUNEL for DNA fragmentation and DAPI for nuclei was performed (FIG. 5A). For apoptosis and proliferation assays, the fixed samples were first probed with anti-α-actinin antibody to identify myocytes (Sigma). Nuclear staining for DNA fragmentation was performed by the terminal deoxynucleotide transferase-mediated dUTP nick end labeling (TUNEL) method (Roche Molecular Biochemicals) for apoptosis. The number of TUNEL positive cells was significantly decreased in the rAAV-GH treated group (17.25±2.58) compared to that of the rAAV-lacZ control (33.25±6.13, FIG. 5B).

Ki-67 staining was performed on heart sections injected with rAAV-LacZ (FIG. 6A) and rAAV-GH (FIG. 6B) using a rabbit polyclonal antibody against Ki-67 (Novocastra Laboratories Ltd., Newcastle, United Kingdom) for cell proliferation followed by DAPI staining (Roche) to count the number of nuclei in peri-infarct area. Ki67 is present only in nuclei of cycling cells as a marker of the late G1-M phase. Expression of Ki67 protein in nuclei of left ventricular myocytes was measured to evaluate whether myocyte proliferation plays a role in the favorable cardiac restructuring of infarct heart following rAAV-GH treatment. In comparison with the lacZ control group, the number of Ki67 positive myocytes increased in the rAAV-GH treated group about 2.0 fold (FIG. 6C, 202±34 versus 410±43). These data demonstrate that expression of hGH promotes favorable cardiac restructuring post-MI.

### Example 8

### Assay for Activation Akt and Stat3 in Heart Post-MI/rAAV Injection

Phosphatidylinositol 3 kinase (PI3K)/Akt pathway is an important anti-apoptotic signaling cascade in cardiac myocytes and JAK2/STAT3 cascade also protects cardiac myocytes from apoptosis. To explore the mechanism of GH effects on protection of myocytes, the affects of growth hormone on STAT3, Akt (anti-apoptosis effectors), caspase 3 (an apoptosis effector), or PCNA (proliferating cell nuclear antigen, a cell cycle protein) in a post-MI heart was analyzed. Proteins from heart lysates were separated by SDS-PAGE, blotted onto nitrocellulose membrane (Millipore), and incubated with polyclonal antibodies to phospho-STAT3, STAT3, phospho-Akt, Akt, PCNA or caspase 3 (Santa Cruz). After washing and incubating with HRP-linked anti-rabbit IgG, immunoreactive proteins were visualized with ECL Plus detection system (Amersham). rAAV-mediated GH expression significantly increased phosphorylation of STAT3 and Akt, and induced PCNA expression. In contrast, GH treated tissues exhibited a significant decrease in caspase 3 activity compared to the lacZ group by Western blot analysis. These data demonstrate a role for hGH in inhibition of apoptosis in the post-MI heart (FIG. 7).

### Example 9

### Assay for Expression of p53 and p21 in Heart Post-MI/rAAV Injection

Recent studies indicate that accumulation of p53 and p21 (WAF1/CIP1) suppress endothelial cell and cardiac myocyte proliferation. RT-PCR was performed on total heart RNA extracted as above to examine the effect of GH overexpression on p53 and p21 in the infarct heart. rAAV-mediated GH expression significantly inhibited mRNA expression of p53 and p21 by about 50% compared to the control lacZ group (FIGS. 8A and B). These data demonstrate that the favorable cardiac restructuring observed in response to expression of hGH post-MI is at least in part due to suppression of p53 and p21 expression.

### Statistical Analysis

The mean and standard error (S.E.) were determined for multiple samples. Unpaired Student's t-test was performed to calculate the statistical significance between the means of two groups. A p value of less than 0.05 was considered significant.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

Methods useful for practicing the methods of the invention are known in the art. See, for example, the following list of publications, each of which is hereby incorporated by reference in its entirety.

1. A method of increasing angiogenesis, cell proliferation, or muscle function; or decreasing apoptosis; or ameliorating cardiac disease in a muscle tissue or a cardiac tissue in a subject in need thereof, the method comprising administering to the subject an effective amount of a recombinant adeno-associated viral vector expressing growth hormone or a fragment or variant thereof, wherein the administration of the viral vector expressing the growth hormone increases at least one of angiogenesis, cell proliferation, or muscle function; or decreases apoptosis; or ameliorates cardiac disease.

2. (canceled)

3. (canceled)

4. (canceled)

5. (canceled)

6. The method of claim 1, wherein the growth hormone is human growth hormone.

7. The method of claim 1, wherein the cardiac disease is selected from the group consisting of myocardial infarction, cardiac ischemia, cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure

8. The method of claim 1, wherein the method increases levels of phosphorylated Akt and Stat-3.

9. The method of claim 1, wherein the method increases levels of nitric oxide synthase, VEGF, bFGF, and angiopoietin.

10. The method of claim 1, wherein the method decreases levels of activated caspase 3.

11. The method of claim 1, further comprising administering to the subject an angiogenic factor or a nucleic acid encoding an angiogenic factor.

12. The method of claim 11, wherein the angiogenic factor is VEGF, IGF-1, or a functional fragment thereof.

13. The method of claim 1, wherein the subject is diagnosed as having a cardiac indication selected from the group consisting of cardiac ischemia, myocardial infarction, cardiomyopathy, and cardiomyositis.

14. A method for preventing, treating or reducing severity of ischemia in a muscle tissue or a cardiac tissue in a subject in need thereof, the method comprising administering to the subject a therapeutically effective amount of a recombinant adeno-associated viral vector encoding growth hormone, wherein administration of the recombinant adeno-associated viral vector encoding growth hormone results in prevention, treatment, or reduction of the severity of ischemia in a muscle tissue or a cardiac tissue in a subject.

15. The method of claim 14, wherein the growth hormone is human growth hormone.

16. The method of claim 1, wherein the subject is diagnosed as having a cardiac indication selected from the group consisting of cardiac ischemia, myocardial infarction, cardiomyopathy, and cardiomyositis.

17. The method of claim 1, wherein the vector is administered by direct injection into a muscle tissue, a cardiac tissue or via a blood vessel supplying the muscle tissue.

18. The method of claim 17, wherein the vector is administered to a plurality of sites at one time.

19. The method of claim 1, wherein the vector is administered at a dose of virus particles/kg subject.

20. The method of claim 1, wherein the vector has an AAV serotype selected from the group consisting of AAV-1, AAV-2, AAV-3, AAV-4, AAV-5, AAV-6, AAV-7, AAV-8, AAV-9, and AAV-10.

21. The method of claim 1, wherein the method further comprises identifying a subject in need of increased angiogenesis, increased cell proliferation, decreased apoptosis, or increased function in muscle tissue or cardiac tissue.

22. The method of claim 1 any of claims 1-21, wherein the method further comprises obtaining a rAAV-GH vector.

23. The method of claim 1, wherein the effect of the viral vector is sustained.

24. The method of claim 1, wherein the muscle tissue or cardiac tissue is ischemic tissue.

25. The method of claim 1, wherein angiogenesis in treated tissue is increased at least 10% as compared to control tissue.

26. The method of claim 1, wherein cell proliferation in treated tissue is increased at least 10% as compared to control tissue.

27. The method of claim 1, wherein apoptosis in treated tissue is decreased at least 10% as compared to control.

28. The method of claim 1, wherein cardiac function is improved at least 10% as compared to control.

29. A recombinant muscle cell or cardiac cell comprising a recombinant adeno-associated viral vector comprising a nucleic acid sequence encoding a human growth hormone polypeptide, variant, or a fragment thereof.

30. The cell of claim 29, wherein the growth hormone is human growth hormone.

31. The cell of claim 29, wherein the cell is a human cell.

32. The cell of claim 29, wherein the cell is stably transduced.

33. The cell of claim 29, wherein the vector is a replication defective adeno-associated viral vector.

34. The cell of claim 29, wherein the vector has an AAV serotype selected from the group consisting of AAV-1, AAV-2, AAV-3, AAV-4, AAV-5, AAV-6, AAV-7, AAV-8, AAV-9, and AAV-10.

35. The cell of claim 29, wherein the cell is in vivo.

36. (canceled)

37. (canceled)

38. (canceled)

39. (canceled)

40. (canceled)

41. (canceled)

42. A kit for transducing a cardiac tissue, the kit comprising an adeno-associated viral vector comprising a nucleic acid sequence encoding growth hormone.

43. The kit of claim 42, wherein the growth hormone is human growth hormone or a functional fragment thereof.

44. The kit of claim 42, wherein the kit further comprises directions for administering the vector to a cardiac cell.

45. The method of claim 14, wherein the vector is administered by direct injection into a muscle tissue, a cardiac tissue or via a blood vessel supplying the muscle tissue.

46. The method of claim 45, wherein the vector is administered to a plurality of sites at one time.
